(19) United States
(12) Patent Application Publication
(10) Pub. No.: US 2025/0017694 A1
(43) Pub. Date: Jan. 16, 2025

(54) Geometric Deep Learning for Setups and Staging in Clear Tray Aligners

(71) Applicant: Solventum Intellectual Properties Company, Maplewood, MN (US)

(72) Inventors: Jonathan D. Gandrud, Woodbury, MN (US); Seyed Amir Hossein Hosseini, Saint Paul, MN (US); Wenbo Dong, Lakeville, MN (US)

(21) Appl. No.: 18/713,011

(22) PCT Filed: Nov. 29, 2022
(86) PCT No.: PCT/IB2022/061551

§ 371 Date: May 23, 2024

Related U.S. Application Data
(60) Provisional application No. 63/264,914, filed on Dec. 3, 2021.

(51) Int. Cl. A61C 7/00; A61C 7/08; G16H 50/50

(52) U.S. Cl. CPC A61C 7/002; A61C 7/08; G16H 50/50

(57) ABSTRACT

Systems and techniques are described for training and using a generative adversarial network (GAN) to produce intermediate stages and final setups for clear tray aligners (CTAs) including receiving, by one or more computer processors, a first digital representation of a patient's teeth, using, by the one or more computer processors and to determine a prediction for one or more tooth movements, a generator that is a neural network included in a GAN and that has been trained to predict one or more tooth movements, and producing, by the one or more processors, an output state that includes at least one of a final setup and one or more intermediate stages.

### TECHNICAL FIELD

This disclosure relates to configurations and training of neural networks to improve the accuracy of automatically generated clear tray aligner (CTA) devices used in orthodontic treatments.

### BACKGROUND

Intermediate staging of teeth from a malocclusion stage to a final stage requires determining accurate individual teeth movements in a way that teeth are not colliding with each other, the teeth move toward their final state, and the teeth follow optimal and preferably short trajectories. Since each tooth has six degrees-of-freedom and an average arch has about fourteen teeth, finding the optimal teeth trajectory from initial to final stage is a large and complex problem.

Previous approaches for automating the production of CTAs involved the use of certain rules or metrics to quantify the state of a set of teeth for realignment using one or more CTA devices. Other approaches attempted to use machine learning techniques to generate the CTA devices, but with mixed results. As a result, there is a need for better machine learning models and training approaches to improve the systems that automate the production of CTAs.

### SUMMARY

The present disclosure describes systems and techniques for training and using a generative adversarial network (GAN) to produce intermediate stages and final setups for CTAs. In a first aspect, a first computer-implemented method for generating setups for orthodontic alignment treatment is described including the steps of receiving, by one or more computer processors, a first digital representation of a patient's teeth, using, by the one or more computer processors and to determine a prediction for one or more tooth movements for a final setup, a generator that is a neural network included in a generative adversarial network GAN and that has been initially trained to predict one or more tooth movements for a final setup, further training, by the one or more computer processors, the GAN based on the using, and where the training of the GAN is modified by performing operations including predicting, by the generator, one or more tooth movements for a final setup based on the first digital representation of the patient's teeth, determining, by a discriminator that is also a neural network configured to distinguish between predicted tooth movements and reference tooth movements and is also part of the GAN, whether a representation of the one or more tooth movements predicted by the generator is distinguishable from a representation of one or more reference tooth movements, and modifying one of the neural networks for at least one of the generator and the discriminator based on the determination of the discriminator.

The first aspect can optionally include additional features. For instance, the method can produce, by the one or more processors, an output state for the final setup. The method can determine, by the one or more computer processors, a difference between the one or more predicted tooth movements and the one or more reference tooth movements. The determined difference between the one or more predicted tooth movements and the one or more reference tooth movements can be used to modify the training of the generator. Modifying the training of the generator can include adjusting one or more weights of the generator's neural network. The method can generate, by the one or more computer processors, one or more lists specifying elements of the first digital representation of the patient's teeth. At least one of the one or more lists can specify one or more edges in the first digital representation of the patient's teeth. At least one of the one or more lists can specify one or more polygonal faces in the digital representation of the patient's teeth. At least one of the one or more lists can specify one or more vertices in the first digital representation of the patient's teeth. The method can compute, by the one or more computer processors, one or more mesh features. The one or more mesh features can include edge endpoints, edge curvatures, edge normal vectors, edges movement vectors, edge normalized lengths, vertices, faces of associated three-dimensional representations, voxels, and combinations thereof. The method can generate, by the one or more computer processors, a digital representation predicting the position and orientation of the patient's teeth based on the one or more predicted tooth movements. The method can generate, by the one or more computer processors, a digital representation of the patient's teeth based on the one or more reference tooth movements. Determining, by the discriminator whether a representation of the one or more tooth movements predicted by the generator is distinguishable from a representation of one or more reference tooth movements can include the steps of receiving the representation of the one or more tooth movements predicted by the generator, the representation of the one or more reference tooth movements, and the first digital representation of the patient's teeth, comparing the representation of the one or more tooth movements predicted by the generator, the representation of the one or more reference tooth movements, wherein the comparison is based at least in part on the first digital representation of the patient's teeth, and determining, by the one or more computer processors, a probability that the representation of the one or more tooth movements predicted by the generator is the same as the representation of one or more reference tooth movements.

In a second aspect, a second computer-implemented method for generating setups for orthodontic alignment treatment is described including the steps of receiving, by one or more computer processors, a first digital representation of a patient's teeth, and a representation of a final setup, using, by the one or more computer processors and to determine a prediction for one or more tooth movements for one or more intermediate stages, a generator that is a neural network included in a generative adversarial network (GAN) and that has been initially trained to predict one or more tooth movements for one or more intermediate stages, further training, by the one or more computer processors, the GAN based on the using, wherein the training of the GAN is modified by performing operations including predicting, by the generator, one or more tooth movements for at least one intermediate stage based on the first digital representation of the patient's teeth, determining, by a discriminator that is also a neural network configured to distinguish between predicted tooth movements and reference tooth movements and is also part of the GAN, whether a representation of the one or more tooth movements predicted by the generator is distinguishable from a representation of one or more reference tooth movements, and modifying one of the neural networks for at least one of the generator and the discriminator based on the determination of the discriminator. The second aspect can also include one or more of the optional features described above in reference to the first aspect.

In a third aspect, a third computer-implemented method for generating setups for orthodontic alignment treatment is described including the steps of receiving, by one or more computer processors, a first digital representation of a patient's teeth, using, by the one or more computer processors and to determine a prediction for one or more tooth movements, a generator that is a neural network included in a generative adversarial network (GAN) and that has been trained to predict one or more tooth movements, and producing, by the one or more processors, an output state that includes at least one of a final setup and one or more intermediate stages, where the GAN has been trained using the operations including predicting, by the generator, one or more tooth movements based on the first digital representation of the patient's teeth, determining, by a discriminator that is also a neural network configured to distinguish between predicted tooth movements and reference tooth movements and is also part of the GAN, whether a representation of the one or more tooth movements predicted by the generator is distinguishable from a representation of one or more reference tooth movements, and modifying one of the neural networks for at least one of the generator and the discriminator based on the determination of the discriminator. The third aspect may also include or more of the optional features described above in reference to the first aspect.

### BRIEF DESCRIPTION OF DRAWINGS

FIG. 1 is an example technique that can be used to train machine learning models used to determine final setups for CTAs.

FIG. 2 is an example visualization of the workflow that is performed using the technique shown in FIG. 1.

FIG. 3 is a different view of the technique shown in FIG. 1.

FIG. 4 is an example technique that can be used to train machine learning models used to determine intermediate staging for CTAs.

FIG. 5 is an example visualization of the workflow that is performed using the technique shown in FIG. 4.

FIG. 6 is a different view of the technique shown in FIG. 4.

FIG. 7 is an expanded view of the technique shown in FIG. 1 that focuses on aspects of the technique that uses geometric deep learning.

FIG. 8 shows an example workflow using a U-Net architecture for the generator shown in either FIG. 1 or FIG. 4.

FIG. 9 shows an example U-Net architecture show in in FIG. 8.

FIG. 10 shows an example workflow 1000 for the generator 110 shown in either FIG. 1 or FIG. 4.

FIG. 11 show an example pyramid encoder-decoder shown in FIG. 10.

FIG. 12 shows an example encoder shown in FIGS. 8 and 10.

FIG. 13 shows an example processing unit that operates in accordance with the techniques of the disclosure.

### DETAILED DESCRIPTION

Clear tray aligners, or CTAs, are a series of orthodontic molds that are used to realign the positioning and/or orientation of any number of the patient's teeth over the course of treatment. As the patent's teeth conform to one tray, or mold, the existing tray can be replaced with the next tray in the sequence to achieve the desired results. CTAs can be made of various materials, but as the name indicates, they are generally clear, so the trays can be worn throughout the day to achieve the desired affect without being overly distracting, cosmetically. Used herein, a “final setup” means a target arrangement of teeth that corresponds to the final CTA in the sequence (i.e., that represents the final desired alignment of the patient's teeth). “Intermediate stages” is used herein to identify an intermediate arrangement of teeth that corresponds to other trays in the sequence that are used to reach the final setup.

Automation tools have been developed for the creation of digital final setups and intermediate stages that can be used to generate the physical trays that represent the intermediate stages and final setups. In one known implementation, a landmark-based anatomy-driven approach was used, which attempted to quantify the state of a set of teeth according to certain metrics or rules to determine the digital representations of the intermediate stages and final setups. In another implementation, a neural network was used for the generation of the digital representation for the final setups and intermediate stages.

In working with these solutions, however, areas of improvement have been identified. One area of improvement is that models implemented by the above techniques may occasionally not generate all of the tooth movements that would be required to position the patient's teeth as desired. For instance, it has been observed that computed tooth movements may occasionally cause one or more teeth to continue to overlap, to name one example. This can ultimately result in a sequence of aligners that does not produce the desired cosmetic outcome because certain teeth may still overlap even after the final setup is worn by the patient. To address identified shortcomings in the trained models, the digital representations may need to be processed additional times, resulting in additional computational overhead, which unnecessarily consumes computing resources. Even then, it may not be possible to fix the digital representations and so human intervention may be needed to correct the resulting outputs of these systems and techniques. In other words, advantages of the instant disclosure provide for better trained systems which result in more accurate digital representations and improved automation of the underlying system, to name two examples. In short, systems practicing the disclosed techniques are better trained and produce more accurately generated digital representations of the respective intermediate stages and final setups, and do so in a shorter duration of time.

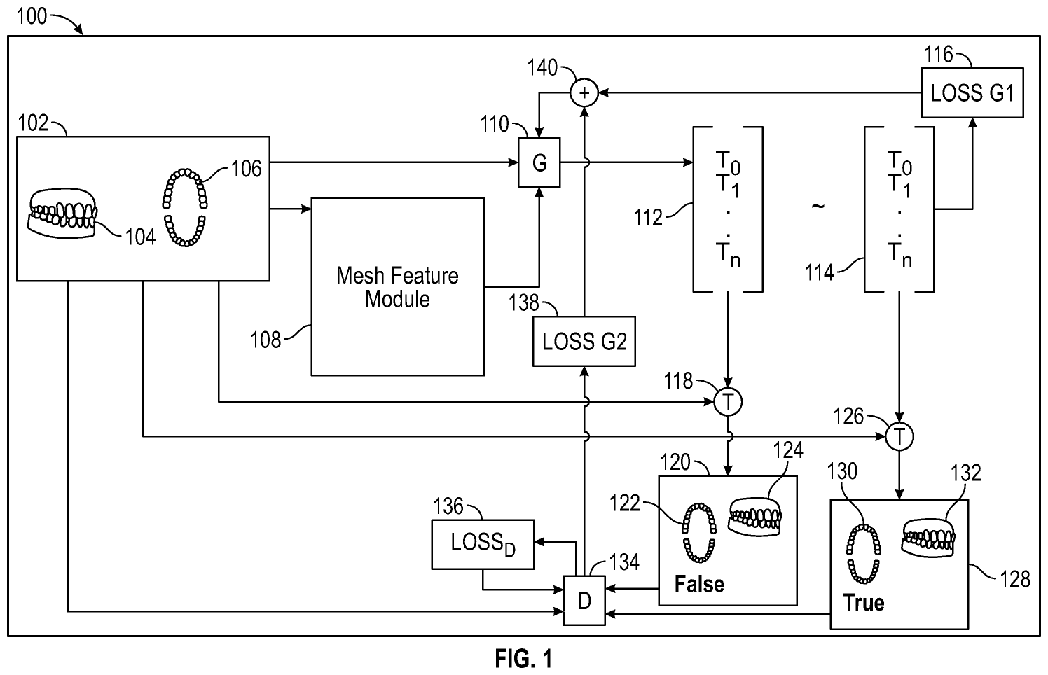

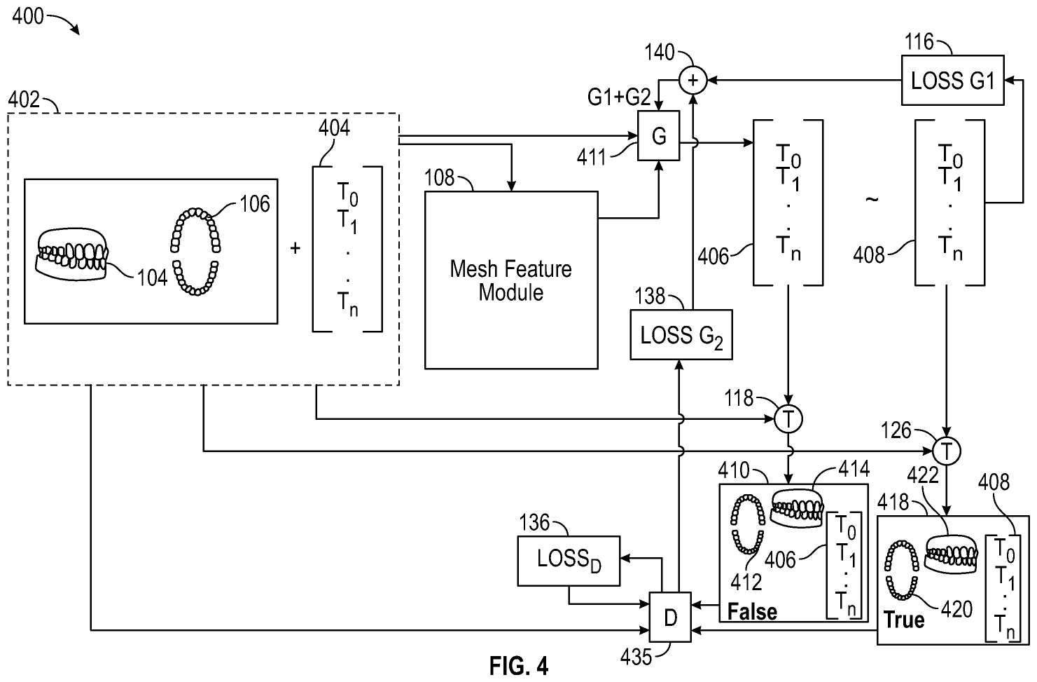

FIG. 1 is an example technique 100 that can be used to train machine learning models used to determine final setups for CTAs. As will be discussed in more detail below, the technique 100 can be implemented on computer hardware to achieve the desired results. A receiving module 102 receives patient case data. In general, the patient case data represents a digital representation of the patient's mouth. As illustrated, patient case data received by module 102 can be received as either malocclusion arches 106 (e.g., a 3-dimensional (“3D”) meshes that represent the upper and lower arches of the patient's teeth), the malocclusion arches 106 as shown, but arranged in a “bite” position 104, where the upper and lower arches are engaged with each other, or a combination of the two. According to particular implementations, the 3D mesh representing bite position 104 may also include 3D mesh geometry for the patient's gingival tissue (i.e., gums) in addition to the mesh data for the patient's teeth. Portions of the description of FIG. 1 are presented as being agnostic to the type of training being performed (i.e., training for final setup or training for intermediate stages). It should be understood, however, that when referencing mesh transformations training resulting in analyzing those transformations, technique 100 is intended to operate for the purpose of training neural networks to more accurately and quickly generate final setups. Training for intermediate stages is described in reference to FIG. 4.

It should be understood that according to particular implementations, bite position geometry 104 and malocclusion arch geometry 106 may include or be otherwise defined by the same or similar 3D geometries but arranged in a particular configuration. That is, in some situations, bite position geometry 104 contains the same underlying mesh data-arranged or otherwise drawn to represent a bite configuration—as is contained in the malocclusion arch geometries 106. Thus, for example, if the receiving module 102 receives only the malocclusion arch geometries 106, the receiving module 102 may automatically generate the bite position geometry 104. Conversely, if the receiving module 102 receives only the bite position geometry 104, the receiving module 102 may automatically generate the malocclusion arch geometry 106. Used herein, “3D mesh,” and “3D geometry” are used interchangeably to reference the 3D digital representations. That is, it should be understood, without loss of generality, that there are various types of 3D representations. One type of 3D representation may comprise of a 3D mesh, a 3D point cloud, a voxelized geometry (i.e., collection of voxels), or other representations which are described by mathematical equations. Although the term “mesh” is used frequently throughout this disclosure, the term should be understood, in some implementations, to be interchangeable with other types of 3D representations.

Each aspect of the various setups prediction implementations described herein is applicable to the fabrication of clear tray aligners and indirect bonding trays. The various setups prediction implementations may also be applicable to other products that involve final teeth poses. A pose comprises at least one of a position (or location) and a rotation (or orientation).

A 3D mesh is a data structure which describes the geometry (or shape) and structure of an object, such as a tooth, a hardware element or the patient's gum tissue. A 3D mesh comprises of mesh elements such as vertices, edges and faces. In some implementations, mesh elements may include voxels, such as in the context of sparse mesh processing operations. Various spatial and structural features may be computed for these mesh elements and be inputted to the predictive models of this disclosure, with the advantage of improving the ability of those models to make accurate predictions.

Mesh feature module 108 can use the patient case data received by receiving module 102 and compute a number of features related to the 3D meshes 104 and 106. In general, technique 100 is most concerned with optimizing the 3D geometry related to the patient's teeth and less concerned with optimizing the 3D geometry related to the patient's gingival tissue. As a result, mesh feature module 108 is configured to compute features for each tooth present in the corresponding 3D geometry. According to particular implementations, the mesh feature module 108 can compute one or more of: edge midpoints, edge curvatures, edge normal vectors, edge normalization vectors, edge movement vectors, and other information pertaining to each tooth in the 3D meshes 104 and 106. According to particular implementations, mesh feature module 108 may or may not be utilized. That is, it should be appreciated that the computation of any of the edge midpoints, edge curvatures, edge normal vectors, and edge movement vectors for each tooth in the 3D meshes 104 and 106 is optional. One advantage of using the mesh feature module 108 is that a system utilizing mesh feature module 108 can be trained more quickly and accurately, but the technique 100 nevertheless performs better than existing techniques without the use of the mesh feature module 108. Another advantage of using 3D meshes over traditional approaches, is that errors incurred by mapping two-dimensional results back into 3D spaces are not present in the present disclosure. Therefore, operating directly in 3D improves the underlying accuracy of the machine learning model and the results generated therefrom.

A 3D mesh comprises of edges, vertices and faces. Though interrelated, these three types of data are distinct. The vertices are the points in 3D space that define the boundaries of the mesh. These points would be described as a point cloud without the additional information about how the points are connected to each other, the edges. An edge comprises of two points and can also be referred to as a line segment. A face comprises of edges and vertices. In the case of a triangle mesh, a face comprises of three vertices, where the vertices are interconnected to form three contiguous edges. Some meshes may contain degenerate elements, such as non-manifold geometry, which must be removed before processing can proceed. Other mesh pre-processing operations are possible. 3D meshes are commonly formed using triangles, but may in other implementations be formed using quadrilaterals, pentagons, or some other n-sided polygon. In some implementations, a 3D mesh may be converted to one or more voxelized geometries (i.e., comprising voxels), such as in the case that sparse processing is performed.

The techniques of this disclosure which operate on 3D meshes may receive as input one or more tooth meshes (e.g., arranged in one or more dental arches). Each of these meshes much undergo pre-processing before being input to the predictive architecture (e.g., including at least one of an encoder, decoder, pyramid encoder-decoder and U-Net). This pre-processing includes the conversion of the mesh into lists of mesh elements, such as vertices, edges, faces or in the case of sparse processing—voxels. For the chosen mesh element type or types, (e.g., vertices), feature vectors are generated. In some examples, one feature vector is generated per vertex of the mesh. Each feature vector may contain a combination of spatial and structural features, as specified in the following table:

TABLE 1

| Element | Spatial Features | Structural Features |
|---|---|---|
| Edges | XYZ position of an edge | Edge curvature (depends on a |
| | midpoint, XYZ positions of | connectivity neighborhood, |
| | the edge vertices, and the | average curvature of two |
| | normal vector at an edge | vertices), dihedral angles, edge |
| | midpoint (average of the | length, density measure such as |
| | normal vectors of two | a count of incident edges (i.e., a |
| | vertices). | count of the other neighboring |
| | | edges which share the vertices |
| | | of that edge). |
| Faces | XYZ position of a face | Face curvature (average |
| | centroid, surface normal | curvature of the vertices of the |
| | vector. | face), area, density measure |
| | | such as count of adjacent faces |
| | | (i.e., which share at least one |
| | | edge with the face). |
| Vertices | XYZ position, normal | Vertex curvature, density |
| | vector (weighted average of | measure such as the count of |
| | connecting faces for the | vertices within a radius of the |
| | vertex). | vertex, density measure such as |
| | | the count of incident edges. |
| Voxels | XYZ centroid. | Volume, [height × depth × width] |
| | | dimensions, density measure |
| | | such as a count of contained |
| | | vertices, density measure such as |
| | | count of intersected faces, |
| | | density measure such as count of |
| | | intersected edges. |

Consistent with the above descriptions, a voxel may also have features which are computed as the aggregates of the other mesh elements (e.g., vertices, edges and faces) which either intersect the voxel or, in some implementations, are predominantly or fully contained within the voxel. Rotating the mesh does not change structural features but may change spatial features. And, as already described, the term mesh should be considered in a non-limiting sense to be inclusive of 3D mesh, 3D point cloud and 3D voxelized geometry. In some implementations, apart from mesh element features, there are alternative methods of describing the geometry of a mesh, such as 3D keypoints and 3D descriptors. Examples of such 3D keypoints and 3D descriptors are found in “TONIONI A, et al. in ‘Learning to detect good 3D keypoints.’, Int J Comput Vis. 2018 Vol. 0.126, pages 1-20.”. 3D keypoints and 3D descriptors may, in some implementations, describe extrema (either minima or maxima) of the mesh surface.

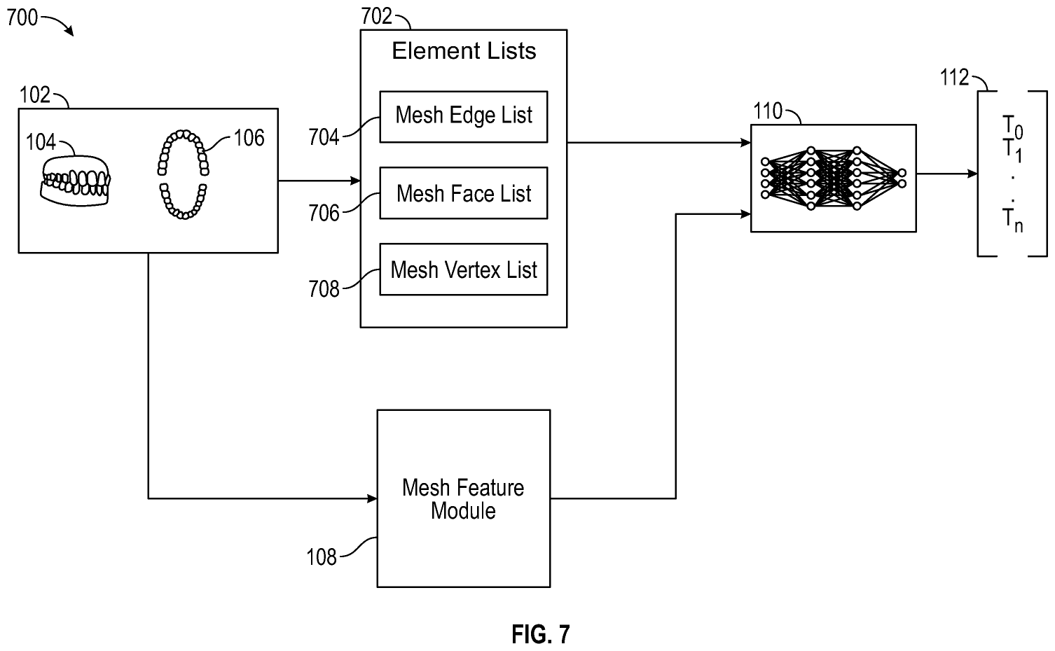

Technique 100 also leverages a generative adversarial network (“GAN”) to achieve certain aspects of the improvements. In general, a GAN is a machine learning model where two neural networks “compete” against each other to provide predictions, these predictions are evaluated, and the evaluations of the two models are used to improve the training of each. As shown in FIG. 1, the two neural networks of the GAN are a generator 110 and a discriminator 134. Generator 110 receives input (e.g., one or more of the bite positions 104, malocclusion arches 106, and mesh features determined by mesh feature module 108). The generator 110 uses the received input to determine predicted tooth movements 112 for each tooth mesh. In some implementations, the generator 110 may also receive random noise, which can include garbage data or other information that can be used to purposefully attempt to confuse the generator 110. The manner in which the generator 110 determines the predicted tooth movements 112 is described in more detail below in FIGS. 7-12.

As described herein, tooth movements specify one or more tooth transformations that can be encoded in various ways to specify tooth positions and orientations within the setup and are applied to 3D representations of teeth. For instance, according to particular implementations, the tooth positions can be cartesian coordinates of a tooth's canonical origin location which is defined in some semantic context. Tooth orientations can be represented as rotation matrices, unit quaternions, or another 3D rotation representations such as Euler angles with respect to a frame of reference (either global or local). Dimensions are real valued 3D spatial extents and gaps can be binary presence indicators or real valued gap sizes between teeth especially in instances when certain teeth are missing. In some implementations, tooth rotations may be described by 3×3 matrices (or by matrices of other dimensions). Tooth position and rotation information may, in some implementations, be combined into the same transform matrix, for example, as a 4×4 matrix, which may reflect homogenous coordinates. In some instances, affine spatial transformation matrices may be used to describe tooth transformations, for example, the transformations which describe the maloccluded pose of a tooth, an intermediate pose of a tooth and/or a final setup pose of a tooth. Some implementations may use relative coordinates, where setup transformations are predicted relative to malocclusion coordinate systems (i.e., a malocclusion-to-setup transformation is predicted instead of a setup coordinate system directly). Other implementations may use absolute coordinates, where setup coordinate systems are predicted directly for each tooth. In the relative mode, transforms can be computed with respect to the centroid of each tooth mesh (vs the global origin), which is termed “relative local.” Some of the advantages of using relative local coordinates include eliminating the need for malocclusion coordinate systems (landmarking data) which may not be available for all patient case datasets. Some of the advantages of using absolute coordinates include simplifying the data preprocessing as mesh data are originally represented as relative to the global origin.

After the predicted tooth movements 112 are determined by the generator 110, the generator 110 can be trained. For instance, in one implementation, each of the predicted tooth movements 112 is compared to the corresponding ground truth tooth movements 114 for each tooth mesh. For instance, the predicted tooth movements 112 for the canine tooth corresponding to number twenty-seven of the international tooth number system would be compared with the ground truth tooth movements 114 for the same canine tooth. A ground truth tooth movement is a tooth movement that has been verified as the correct tooth movement for a particular tooth mesh. In some implementations, the ground truth tooth movements 114 are specified by a human user, such as a dentist or other healthcare provider. In other implementations, the ground truth tooth movements 114 can be generated automatically based on the patient case data or other information provided to a system implementing technique 100.

The difference between the predicted tooth movements 112 and the ground truth tooth movements 114 can be used to compute one or more loss values G1 116. For instance, G1 116 can represent a regression loss between the predicted tooth movements 112 and the ground truth tooth movements 114. That is, according to one implementation, loss G1 116 reflects a percentage by which predicted tooth movements 112 deviate from the ground truth tooth movements 115. That said, generator loss G1 116 can be an L2 loss, a smooth L1 loss, or some other kind of loss. According to particular implementations, an L1 loss is defined as L1=Σᵢ₌₀ⁿ|Pᵢ−Gᵢ|, where P represents the predicted tooth movements 112 and G represents the ground truth tooth movements 114. Tooth movements may be embodied by at least one of a transformation matrix (e.g., an affine transform), a quaternion and a translation vector. According to particular implementations, an L2 loss can be defined as L2=Σᵢ₌₀ⁿ (Pᵢ−Gᵢ)², again where P represents the predicted tooth movements 112 and G represents the ground truth tooth movements 114. In addition, and as will be described in more detail below, the loss values G1 116 can be provided to the generator 110 to further train the generator 110, e.g., by modifying one or more weights in the generator 110's neural network to train the underlying model and improve the model's ability to generate predicted tooth movements 112 that mirror or substantially mirror the ground truth tooth movements 114.

Referring again to the predicted tooth movements 112, each one of the predicted tooth movements 112 is represented by one or more transformations to a respective tooth mesh. For instance, in one implementation, each one of the predicted tooth movements 112 is represented by a six-element rotation vector transform and a 3-element translation vector. In this implementation, the six-element rotation vector represents one or more rotations performed on a respective tooth to modify its rotation within the 3D geometry while the three-element translation vector describes the respective position of each tooth in the 3D geometry using X, Y, and Z coordinates. In other implementations, each one of the predicted tooth movements 112 is represented by a seven-element vector: four elements to describe the quaternion rotation, and three elements to describe the position using X, Y, and Z coordinates. By generating both the rotation and translation predictions as part of determining predictive tooth movements, additional advantages can be realized over existing systems. For instance, it has been observed that generating both translation and rotation predictions together in the same transform improves accuracy over a system that attempts to combine separately predicted translation and rotation predictions, separately or otherwise after one of the translation or rotation prediction was determined.

Using mesh transformers 118 and 126, the technique 100 then transforms the tooth meshes, corresponding to 3D meshes 104 and 106, using the predicated tooth movements 112 and the ground truth tooth movements 114, respectively. That is, the respective transformations are applied to the 3D geometries to modify the 3D geometries to correspond to the specified movements. For instance, in reference to the predicted tooth movements 112 and in implementations that are represented by a seven-element vector, the tooth mesh is rotated using the specified quaternion rotation of the predicted tooth movements 112 for that tooth mesh in 3D meshes 104 and 106 and the mesh's X, Y and Z coordinates are modified to equal the X, Y, and Z coordinates of the predicated tooth movements 112 for that tooth mesh in 3D meshes 104 and 106. Likewise, ground truth tooth movements 114 can be applied to the 3D meshes 104 and 106 to produce a ground tooth movement for each tooth in the 3D meshes 104 and 106.

These transformations result in modified 3D geometries that correspond to a predicted tooth movement representation 120 and a ground truth tooth movement representation 128. According to particular implementations, both the predicted tooth movement representation 120 and ground truth tooth movement representation 128 can include bite position 3D geometries 124 and 132, respectively, and malocclusion arch 3D geometries 122 and 130, respectively. That is, the predicated tooth movement representation 120 can be represented by a bite position mesh 124 and one or more malocclusion arch meshes 122 that correspond to changes in bite position mesh 104 and malocclusion arch meshes 106 as specified by the predicted tooth movement transformations 112. Likewise, the ground truth tooth movement representation 128 can be represented by bite position mesh 132 and one or more malocclusion arch meshes 132 as specified by the ground truth tooth movement transformations 114.

Additionally, the predicted tooth movement representation 120 and ground truth tooth movement representation 128 can be flagged or otherwise annotated to indicate whether the representation corresponds to a ground truth transformation. For instance, in one implementation, the predicted tooth movement representation 120 is assigned a value of “false” to indicate that it does not correspond to the ground truth tooth movements 114 while the ground truth tooth movement representation 128 is assigned a value of “true.”

According to particulate implementations, the representations 120 and 128 are provided as inputs to the discriminator 134. In addition, according to particular implementations, mesh geometries 104 and 106 are also provided to the discriminator 134. That said, the information pertaining to representations 120 and 128 and meshes 104 and 106 may also be provided to discriminator 134 in other ways. Specifically, the discriminator 134 need not receive transformed meshes (i.e., representations 120 and 128). Instead, the discriminator 134 can receive the starting mesh geometries 104 and 106 and the transformations 112 and 114. According to another implementation, instead of the transformations 112 and 114, the discriminator 134 can receive a list of one or more movements that are applied to each element in the meshes 104 and 106. That is, the discriminator 134 can receive various representations of the data corresponding to meshes 104 and 106, the transformations 112 and 114, and the representations 120 and 128. In general, the discriminator 134 is configured to determine when an input is generated from the predicated tooth movements 112 or when an input is generated from the ground truth tooth movement representation 128. For instance, in one implementation, the discriminator 134 may output an indication of “false” when the discriminator 134 determines that the input was generated from the predicated tooth movements 112 and may output an indication of “true” when the input was generated from ground truth tooth movements 114.

The discriminator 134 can be initially trained in a variety of ways. For instance, the discriminator 134 can be configured as an encoder-a specific kind of neural network-which in some situations, such as the ones described herein, can be configured to perform validation. For instance, the initial encoder included in the discriminator 134 can be configured with random edge weights. Using backpropagation, the encoder—and thereby the discriminator 134—can be successively refined by modifying the values of the weights to allow the discriminator 134 to more accurately determine which inputs should be identified as “true” ground truth representations and which inputs should be identified as “false” ground truth representations. In other words, while the discriminator 134 can be initially trained, the discriminator 134 continues to evolve/be trained as technique 100 is performed. And like generator 110, with each execution of technique 100 the accuracy of the discriminator improves. Although as understood by a person of ordinary skill in the art the improvements to the discriminator 134 will reach a limit by which the discriminator 134's accuracy does not statistically improve, at which time the discriminator's 134 training is considered complete.

After the discriminator 134 generates an output, the technique 100 then compares the output of the discriminator 134 against the input to determine whether the discriminator accurately distinguished between the predicted tooth movement representation 120 and ground truth tooth movement representation 128. For instance, the output of the discriminator 134 can be compared against the annotation of the representation. If the output and annotation match, then the discriminator 134 accurately predicted the type of input that the discriminator 134 received. Conversely, if the output and annotation do not match, then the discriminator 134 did not accurately predict the type of input that the discriminator 134 received. In some implementations, and like the generator 110, the discriminator 134 may also receive random noise, purposefully attempting to confuse the discriminator 134.

In addition, and according to particular implementations, the discriminator 134 may generate additional values that can be used to train aspects of the system implementing technique 100. In one example, the discriminator 134 may generate a discriminator loss value 136, which reflects how accurately the discriminator 134 determined whether the inputs corresponded to the predicted tooth movement representation 120 and/or ground truth tooth movement representation 128. According to particular implementations, the discriminator loss 136 is larger when the discriminator 134 is less accurate and smaller when the discriminator 134 is more accurate in its predictions. In another example, the discriminator 134 may generate a generator loss value G2 138. According to particular implementations, while not directly inverse to discriminator loss 136, generator loss value G2 138 generally exhibits an inverse relationship to discriminator loss 136. That is, when discriminator loss 136 is large, generator loss G2 138 is small and when discriminator loss 136 is small, generator loss G2 138 is large. In some implementations, discriminator loss 136 may be determined using a binary cross entropy loss function that is calculated for both “true” and “false” models. In some implementations, generator loss may be composed of two losses: 1) the first loss is the generator loss G2 138 as determined by the discriminator (hence a binary cross entropy may be used); and 2) the second loss may be implemented by an l1-norm or mean square error that measures the difference between the desired output and the actual output of the generator 110, e.g., as specified by generator loss G1 116.

In other words, and as illustrated in FIG. 1, generator loss G2 138 can be added to generator loss G1 116 using a summation operation 140. And the summed value of generator loss G1 116 and G2 138 can be provided to generator 110 for the purposes of training generator 110. That said, it should be appreciated that the computation of the generator loss G1 116 is not necessary to the training of the GAN. In some implementations, it may be possible to train either the generator 110 or the discriminator 134 using only a combination of generator loss G2 138 and discriminator loss 136. But like other optional aspects of this disclosure, using the generation loss G1 116 can be utilized to more quickly train the generator 134 to produce more accurate predictions. Additional aspects of technique 100 will be made apparent as part of the discussion of the subsequent FIGS.

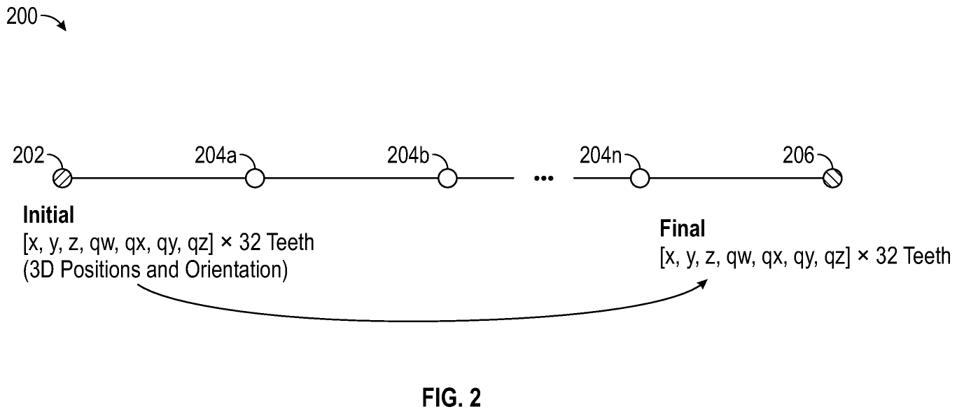

FIG. 2 is an example visualization of the workflow 200 that is performed using the technique 100 shown in FIG. 1. As should be appreciated by the description of FIG. 1, at the first step 202 of the workflow 200, initial 3D position and orientation data (e.g., one or more of the bite positions 104, malocclusion arches 106) is received in the form of one or more 3D geometries, and the technique 100 computes final position and orientation information at step 206 of the workflow 200. Additional steps 204a to 204n are also shown in workflow 200. In general, however, these steps of the workflow are purposefully omitted when determining final setups as described in reference to FIG. 1. Instead, steps 204a-204n can be used to generate intermediate stages, which are described in more detail below in reference to FIGS. 4-6.

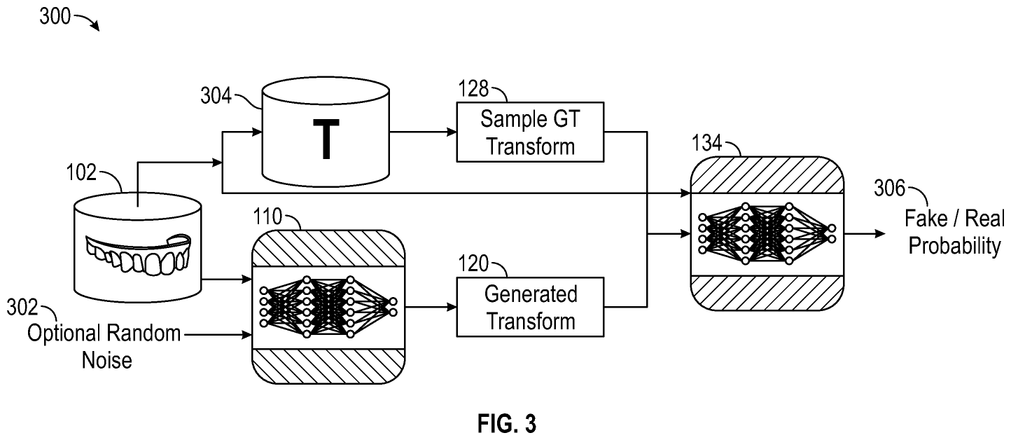

FIG. 3 is a different view of the technique 100 shown in FIG. 1 (referred herein as technique 300). According to particular implementations, technique 300 first accesses patient data, such as patient data received by module 102. At step 302, the processor performing technique 300 can optionally generate random noise. Next, the processor provides the patent case data and the optional random noise to generator 110. As described above in reference to FIG. 1, the processor executes instructions that cause the trained generator 110 to generate predicted tooth movements 112, which can be used to determine predicted tooth movement representation 120.

The system performing technique 300 can also access one or more ground truth transformations at step 304 and select one or more sample ground truth transforms 114 that correspond to the selected patient case data received by module 102. As described above in reference to FIG. 1, one or more sample ground truth transforms 114 can be used to generate a ground truth tooth movement representation 128. Next, the system performing technique 300 can provide any of the patient case data received by the receiving module 102, the predicted tooth movement representation 120, and the ground truth tooth movement representation 128 to the discriminator 134.

Next, as described in reference to technique 100, at step 306, the discriminator 134 determines whether the inputs correspond to a ground truth transformation by providing a probability that the input is a real ground truth transformation or a fake ground truth transformation. In some implementations, the probability returned by the discriminator 134 may be in range of zero and one. That is, the discriminator 134 may provide a value approaching zero to indicate a low probability that the input is real (i.e., corresponds to predicted tooth movements 112) or may provide a value approaching one to indicate a high probability that the input is real (i.e., corresponds to ground truth tooth movements 114).

As described above in reference to FIG. 1, the output of the discriminator 134 can be used to train both the discriminator 134 and the generator 110.

FIG. 4 is an example technique 400 that can be used to train machine learning models used to determine intermediate staging for CTAs. As depicted, aspects of technique 400 are similar to technique 100. For instance, technique 400 utilizes a receiving module 402 which receives patient case data. The receiving module 402 operates similarly to receiving module 102, e.g., the receiving module 402 can receive data corresponding to bite positioning geometry and malocclusion arch geometries 106. The receiving module differs from the receiving module 102 in that receiving module 402 is also configured to receive endpoint tooth transformations 404 that correspond to a final setup. According to particular implementations, the endpoint tooth transformations 404 can be predefined or provided as a result of the outcome of performing technique 100.

Technique 400 also uses the mesh feature module 108 that can use the patient case data received by receiving module 102 and compute a number of features related to the 3D meshes 104 and 106 as described above in reference to FIG. 1. Like technique 100, technique 100 also leverages a generative adversarial network (“GAN”) to achieve certain aspects of the improvements as described throughout this disclosure. Technique 400, however, uses a generator 411 and a discriminator 435 that are used differently than the generator 110 and the discriminator 134 as described above in reference to FIG. 1. For example, generator 411 receives input (e.g., one or more of the bite positions 104, malocclusion arches 106, and mesh features determined by mesh feature module 108) and instead of generating predicated tooth movements for final setups, the generator 411 uses the received input to determine predicted intermediate tooth movements 406 for each tooth mesh. According to certain implementations, the predicted intermediate stage tooth movements 406 can be used to determine one or more of the values: 1) in which direction the tooth is moving, 2) how far towards the final state the tooth is located for the present stage, and 3) how the tooth is rotated. But other aspects of generator 411 are the same. For instance, in some implementations, the generator 411 may also receive random noise, which can include garbage data or other information that can be used to purposefully attempt to confuse the generator 411. As a result, it should be appreciated that in many aspects of the disclosure described herein, generator 110 and generator 411 can be used interchangeably. Likewise, discriminator 134 and discriminator 435 can be used interchangeably.

After the predicted intermediate tooth movements 406 are determined by the generator 411, the generator 411 can be trained. For instance, in one implementation, each of the predicted intermediate tooth movements 406 is compared to the corresponding ground truth intermediate tooth movements 408 for each tooth mesh. The comparison that is performed as part of technique 400 is the same as technique 100 as described in reference to FIG. 1.

Similarly, the difference between the predicted intermediate tooth movements 406 and the ground truth intermediate tooth movements 408 can be used to compute one or more loss values G1 116 as described above in reference to technique 100. Likewise, as described in connection to technique 100, the loss values G1 116 can be provided to the generator 411 to further train the generator 411, e.g., by modifying one or more weights in the generator 411's neural network to train the underlying model and improve the model's ability to generate predicted intermediate tooth movements 406 that mirror or substantially mirror the ground truth intermediate tooth movements 408.

Referring again to the predicted intermediate tooth movements 406, each one of the predicted intermediate tooth movements 406 is represented by one or more transformations to a respective tooth mesh. For instance, in one implementation, each one of the predicted intermediate tooth movements 406 is represented by a six-element rotation vector transform and a 3-element translation vector. In this implementation, the six-element rotation vector represents one or more rotations performed on a respective tooth to modify its rotation within the 3D geometry while the three-element translation vector describes the respective position of each tooth in the 3D geometry using X, Y, and Z coordinates. In other implementations, each one of the predicted intermediate tooth movements 406 is represented by a seven-element vector: four elements to describe the quaternion rotation, and three elements to describe the position using X, Y, and Z coordinates.

Using mesh transformers 118 and 126, the technique 400 then transforms the tooth meshes, corresponding to 3D meshes 104 and 106, using the predicated intermediate tooth movements 406 and the ground truth intermediate tooth movements 408, respectively. That is, the respective transformations are applied to the 3D geometries to modify the 3D geometries to correspond to the specified movements. For instance, in reference to the predicted intermediate tooth movements 406 and in implementations that are represented by a seven-element vector, the tooth mesh is rotated using the specified quaternion rotation of the predicted intermediate tooth movements 406 for that tooth mesh in 3D meshes 104 and 106 and the mesh's X, Y and Z coordinates are modified to equal the X, Y, and Z coordinates of the predicated intermediate tooth movements 406 for that tooth mesh in 3D meshes 104 and 106. Likewise, ground truth intermediate tooth movements 114 can be applied to the 3D meshes 104 and 106 to produce a ground tooth movement for each tooth in the 3D meshes 104 and 106.

These transformations result in modified 3D geometries that correspond to a predicted intermediate tooth movement representation 410 and a ground truth intermediate tooth movement representation 418. According to particular implementations, both the predicted intermediate tooth movement representation 410 and ground truth intermediate tooth movement representation 418 can include bite position 3D geometries 414 and 422, respectively, and malocclusion arch 3D geometries 412 and 420, respectively. That is, the predicated intermediate tooth movement representation 410 can be represented by a bite position mesh 414 and one or more malocclusion arch meshes 412 that correspond to changes in bite position mesh 104 and malocclusion arch meshes 106 as specified by the predicted intermediate tooth movement transformations 406. Likewise, the ground truth intermediate tooth movement representation 418 can be represented by bite position mesh 422 and one or more malocclusion arch meshes 420 as specified by the ground truth intermediate tooth movement transformations 408.

Additionally, the predicted intermediate tooth movement representation 410 and ground truth intermediate tooth movement representation 418 can be flagged or otherwise annotated to indicate whether the representation corresponds to a ground truth transformation. For instance, in one implementation, the predicted intermediate tooth movement representation 410 is assigned a value of “false” to indicate that it does not correspond to the ground truth intermediate tooth movements 408 while the ground truth intermediate tooth movement representation 418 is assigned a value of “true.”

The representations 410 and 418 are provided as inputs to the discriminator 134. In addition, according to particular implementations, mesh geometries 104 and 106 are also provided to the discriminator 435. That said, the information pertaining to representations 410 and 418 and meshes 104 and 106 may also be provided to discriminator 435 in other ways. Specifically, the discriminator 435 need not receive transformed meshes (i.e., representations 410 and 418). Instead, the discriminator 435 can receive the starting mesh geometries 104 and 106 and the transformations 406 and 408. According to another implementation, instead of the transformations 406 and 408, the discriminator 435 can receive a list of one or more movements that are applied to each element in the meshes 104 and 106. That is-like the discriminator 134—the discriminator 435 can receive various representations of the data corresponding to meshes 104 and 106, the transformations 406 and 408, and the representations 410 and 418. In according with technique 400, the discriminator 435 is configured to determine when an input is generated from the predicated intermediate tooth movements 406 or when an input is generated from the ground truth intermediate tooth movements 408. For instance, in one implementation, the discriminator 435 may output an indication of “false” when the discriminator 435 determines that the input was generated from the predicated intermediate tooth movements 406 and may output an indication of “true” when the input was generated from ground truth intermediate tooth movements 408.

The discriminator 435 is otherwise largely the same as the discriminator 435 described in reference to FIG. 1. For instance, after the discriminator 435 generates an output, the technique 400 then compares the output of the discriminator 435 against the input to determine whether the discriminator accurately distinguished between the predicted tooth movement representation 410 and ground truth tooth movement representation 418. For instance, the output of the discriminator 435 can be compared against the annotation of the representation. If the output and annotation match, then the discriminator 435 accurately predicted the type of input that the discriminator 435 received. Conversely, if the output and annotation do not match, then the discriminator 435 did not accurately predict the type of input that the discriminator 435 received. In some implementations, and like the generator 411, the discriminator 435 may also receive random noise, purposefully attempting to confuse the discriminator 435.

In addition, and according to particular implementations, the discriminator 435 may generate additional values that can be used to train aspects of the system implementing technique 400. In one example, the discriminator 435 may generate a discriminator loss value 136, which reflects how accurately the discriminator 435 determined whether the inputs corresponded to the predicted intermediate tooth movement representation 410 and/or ground truth intermediate tooth movement representation 418. According to particular implementations, the discriminator loss 136 is larger when the discriminator 435 is less accurate and smaller when the discriminator 435 is more accurate in its predictions. In another example, the discriminator 435 may generate a generator loss value G2 138. According to particular implementations, while not directly inverse to discriminator loss 136, generator loss value G2 138 generally exhibits an inverse relationship to discriminator loss 136. That is, when discriminator loss 136 is large, generator loss G2 138 is small and when discriminator loss 136 is small, generator loss G2 138 is large. In some implementations, discriminator loss 136 may be determined using a binary cross entropy loss function that is calculated for both “true” and “false” models. In some implementations, generator loss may be composed of two losses: 1) the first loss is the generator loss G2 138 as determined by the discriminator (hence a binary cross entropy may be used); and 2) the second loss may be implemented by an l1-norm or mean square error that measures the difference between the desired output and the actual output of the generator 110, e.g., as specified by generator loss G1 116.

In other words, and as illustrated in FIG. 4, generator loss G2 138 can be added to generator loss G1 116 using a summation operation 140. And the summed value of generator loss G1 116 and G2 138 can be provided to generator 411 for the purposes of training generator 411. Additional aspects of technique 400 will be made apparent as part of the discussion of the subsequent FIGS.

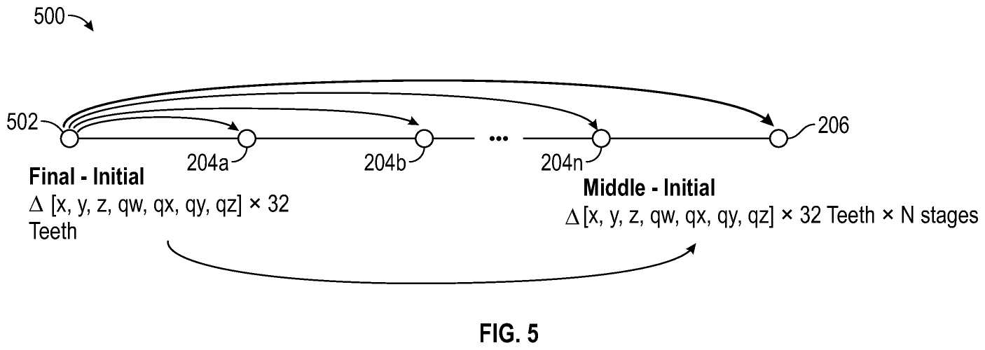

FIG. 5 is an example visualization of a workflow 500 that is performed using the technique 400 shown in FIG. 4. As should be appreciated by the description of FIG. 4, at the first step 502 of the workflow 500, both initial and final 3D position and orientation data (e.g., one or more of the bite positions 104, malocclusion arches 106) is received in the form of one or more 3D geometries, and the technique 400 computes intermediate position and orientation information at steps 204a to 204n in the workflow to generate n intermediate stages for the CTAs.

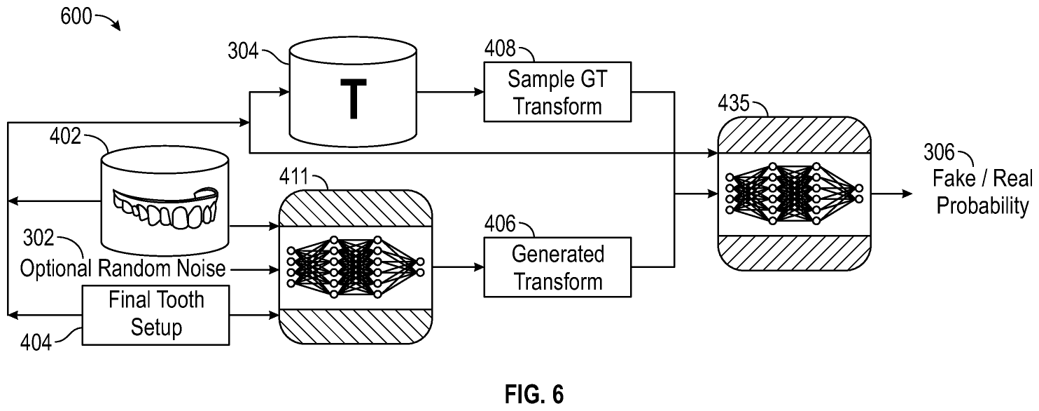

FIG. 6 is different view of the technique 400 shown in FIG. 4 (referred herein as technique 600). According to particular implementations, technique 300 first accesses patient data, such as patient data 402. At step 302, the processor performing technique 600 can optionally generate random noise. Next, the processor provides the patent case data, the final tooth setup 404, and the optional random noise to generator 411. As described above in reference to FIG. 4, the processor executes instructions that cause the trained generator 411 to generate predicted intermediate tooth movements 406, which can be used to determine predicted intermediate tooth movement representation 410.

The system performing technique 600 can also access one or more ground truth transformations at step 304 and select one or more sample ground truth intermediate transforms 408 that correspond to the selected patient case data received by module 102. As described above in reference to FIG. 4, one or more sample ground truth transforms 408 can be used to generate a ground truth tooth movement representation 418. Next, the system performing technique 600 can provide any of the patient case data received by the receiving module 402, the predicted tooth movement representation 410, and the ground truth tooth movement representation 418 to the discriminator 435.

Next, as described in reference to technique 400, at step 306, the discriminator 435 determines whether the inputs correspond to a ground truth transformation by providing a probability that the input is a real ground truth transformation or a fake ground truth transformation. In some implementations, the probability returned by the discriminator 435 may be in range of zero and one. That is, the discriminator 435 may provide a value approaching zero to indicate a low probability that the input is real (i.e., corresponds to predicted intermediate tooth movements 406) or may provide a value approaching one to indicate a high probability that the input is real (i.e., corresponds to ground truth intermediate tooth movements 408).

As described above in reference to FIG. 4, the output of the discriminator 435 can be used to train both the discriminator 435 and the generator 411.

FIG. 7 is an expanded view 700 of the technique 100 shown in FIG. 1 that focuses on aspects of the technique 100 that uses geometric deep learning. According to particular implementations, geometric information pertaining to the 3D meshes 104 and 106 can be identified or otherwise determined by a mesh converter 702. While not included in FIG. 1, it is intended that many implementations of technique 100 would utilize mesh converter 702 because so doing provides various benefits to the technique 100 related to both improving the predictive qualities of the output of the generator 110 as well as improving the training for GAN based on the output of the generator 110 as described above.

As show in FIG. 7, the receiving module 102 can provide the 3D bite position geometry 104 and the 3D malocclusion arch geometries 106 to a mesh converter 702. In general, and according to well established definitions, the 3D geometries 104 and 106 are defined by a collection of vertices, where each pair of vertices specifies an edge of a 3D polygon, and a collection of edges can specify one or more faces (or surfaces) of the 3D geometry. Thus, according to particular implementations, this allows the 3D mesh converter 702 to break down the 3D meshes 104 and 106 into their respective constituent parts.

Stated differently, the 3D mesh converter 702 can extract or otherwise generate various geometric features from the 3D meshes 104 and 106 and those transformed mesh data are then used as an input data to the generator 110. For instance, the 3D mesh converter 702 can generate one or more of the following: one or more mesh edge lists 704, one or more mesh face lists 706, and one or more mesh vertex lists 708.

By providing the generator 110 with this additional information, a number of advantages can be realized. For example, providing this information to the generator 110 allows the generator 110 to generate more accurate predicted tooth movements 112. This in-turn allows for the training a system that implements technique 100 to be improved because both the training of the generator 110 and the training of the discriminator 134 are based, at least in part, on the quality of the predicted tooth movements 112. In short, implementing mesh converter 702 as part of technique 100 can reduce the number of training epochs that the neural networks configured in generator 110 and discriminator 134 must undergo while also improving accuracy. Stated differently, using mesh converter 702 as part of technique 100 allows systems performing technique 100 to conserve computational resources involved in the training process while improving the models that are trained as described.

Furthermore, nothing in the description of the expanded view 700 should be considered limiting. For instance, while the expanded view 700 is shown and described relative to technique 100 presented in FIG. 1, it should be understood that the expanded view 700 may also be used as part of technique 400 shown in FIG. 4. For instance, the receiving module 102 in FIG. 7 can be replaced with receiving module 402 depicted in FIG. 4. This, for example, would allow technique 400 to achieve the same improved utilization of computing resources and model accuracy as described above in connection to technique 100. The only difference being that technique 400 generates predications for intermediate stages while technique 100 generates predictions for final setups. In other words, replacing module 102 with module 402 in FIG. 7 would also cause generator 411 to be substituted for generator 110, and generator 411 produces predicted intermediate tooth movements 406 instead of predicated tooth movements 112. But despite these configuration changes, technique 400 could nevertheless leverage the improvements from geometric deep learning described above.

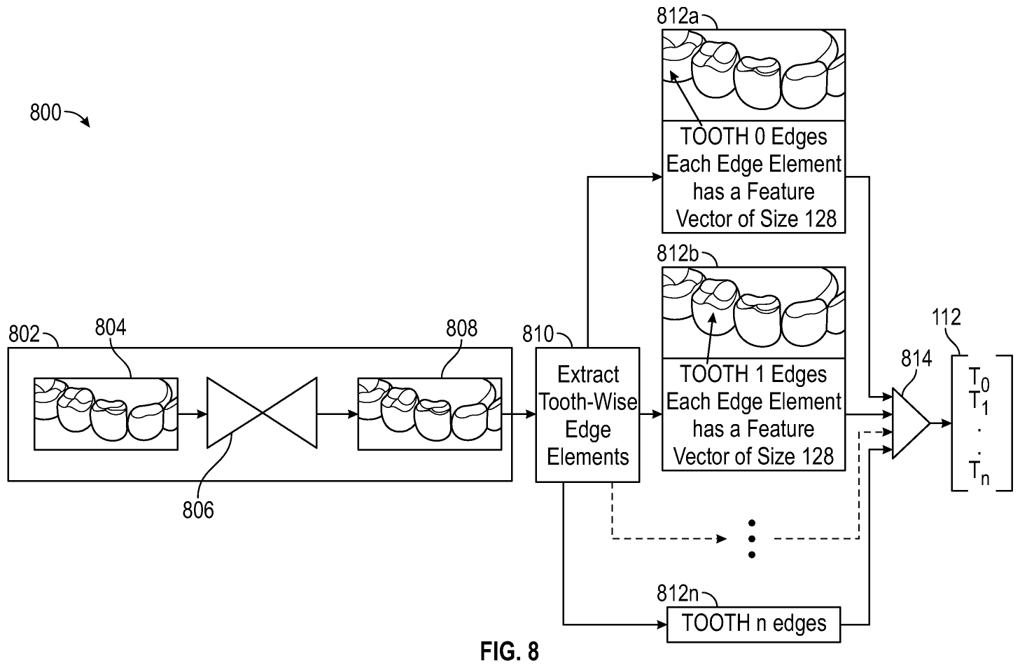

FIGS. 8-12 depict particular aspects of the generator 110, according to particular implementations. In these depicted implementations, the generator 110 can be configured as at least one of a first 3D encoder, a 3D U-Net encoder-decoder or a 3D pyramid encoder-decoder, which is then followed by a second 3D encoder (which may be optionally replaced with a multi-layer perceptron (MLP)). Because the generator can be implemented as one or more neural networks, the generator may contain an activation function. An activation function decides whether or not a neuron in a neural network will fire (e.g., send output to the next layer). Some activation functions may include: binary step functions, and linear activation functions. Other activation functions impart non-linear behavior to the network, including: sigmoid/logistic activation functions, Tanh (hyperbolic tangent) functions, rectified linear units (ReLU), leaky ReLU functions, parametric ReLU functions, exponential linear units (ELU), softmax function, swish function, Gaussian error linear unit (GELU), and scaled exponential linear unit (SELU). A linear activation function may be well suited to some regression applications (among other applications), in an output layer. A sigmoid/logistic activation function may be well suited to some binary classification applications (among other applications), in an output layer. A softmax activation function may be well suited to some multiclass classification applications (among other applications), in an output layer. A sigmoid activation function may be well suited to some multilabel classification applications (among other applications), in an output layer. A ReLU activation function may be well suited in some convolutional neural network (CNN) applications (among other applications), in a hidden layer. A Tanh and/or sigmoid activation function may be well suited in some recurrent neural network (RNN) applications (among other applications), for example, in a hidden layer.

There are multiple optimization algorithms which can be used in the training of the neural networks of this disclosure, including gradient descent (which determines a training gradient using first-order derivatives and is commonly used in the training of neural networks), Newton's method (which may make use of second derivatives in loss calculation to find better training directions than gradient descent, but may require calculations involving Hessian matrices), and conjugate gradient methods (which may have faster convergence than gradient descent, but do not require the Hessian matrix calculations which may be required by Newton's method). The backpropagation algorithm is used to transfer the results of loss calculation back into the network so that network weights can be adjusted, and learning can progress.

In some implementations, the neural networks of this disclosure can be adapted to operate on 3D point cloud data (alternatively on 3D meshes or 3D voxelized geometry). Numerous neural network implementations may be applied to the processing of 3D representations and may be applied to training predictive and/or generative models for oral care applications, including: PointNet, PointNet++, SO-Net, spherical convolutions, Monte Carlo convolutions and dynamic graph networks, PointCNN, ResNet, MeshNet, DGCNN, VoxNet, 3D-ShapeNets, Kd-Net, Point GCN, Grid-GCN, KCNet, PD-Flow, PU-Flow, MeshCNN and DSG-Net.

As described above, each tooth mesh 104 and 106, includes a number of mesh elements, such as edges, faces and vertices. In some implementations, the edges included in the meshes 104 and 106 can be more helpful in generating accurate predictions, although operations could also be performed on faces and vertices. When using edges to make predictions, a feature vector is computed for each edge mesh element. A feature vector may include various 3D geometric representations, such as 3D coordinates of the vertices, or curvatures and midpoints of the edges. Other features are also possible. According to particular implementations, the output of the encoder-decoder structure maintains the same resolution as the input (i.e., the input and the output have the same number of elements). In general, the encoder-decoder structure (either the U-Net architecture or the pyramid architecture) serves to extract high-dimensional features from the tooth mesh, for example, by converting the one or more tooth meshes into representations (which may contain either or both of local and global information about the tooth meshes) which a second encoder may use to generate tooth transforms for either final setups or intermediate stages.

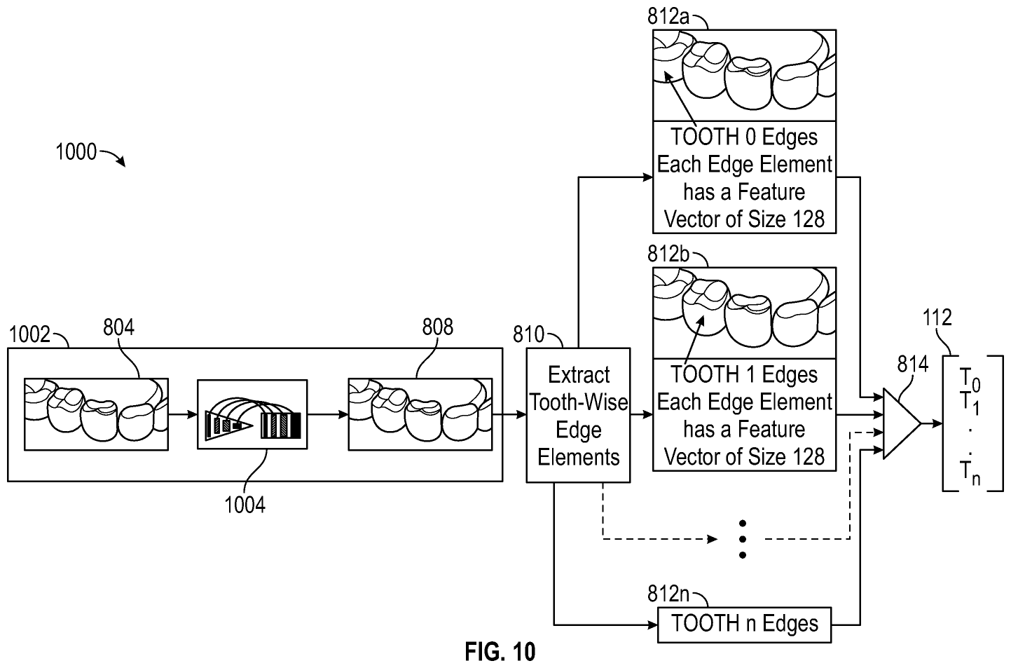

Furthermore, while not expressly shown, additional implementations of FIGS. 8-12 replace the U-Net encoder-decoder 806 or the pyramid encoder-decoder 1004 with an encoder (such as encoder 814 depicted in FIGS. 8 and 10). According to this implementation, the generator 110 operates on lower resolution meshes. That is, the first encoder (not shown, but replacing either the U-Net encoder-decoder 806 or the pyramid encoder-decoder 1004 in FIGS. 8 and 10, respectively) coarsens the resolution of the input geometry that is received by encoder 814. This provides certain advantages including reducing memory consumption of the generator 110. That said, to achieve these improvements additional processing may occur including, but not limited to, maintaining a list of tooth labels for each element (i.e., for each edge, face, or vertex in the 3D geometry).

FIG. 8 shows an example workflow 800 for the generator 110 shown in either FIG. 1 or FIG. 4 and that uses a U-Net architecture. In step 802 of the workflow, input is processed to modify the input into a data format that can be extracted as edge elements. In general, step 802 takes mesh data with a feature vector of a first size, provides to a machine learning model, and the machine learning model generates a feature vector of a second size that corresponds to the mesh data.

As depicted in FIG. 8, the mesh data at step 804 can be any combination of tooth meshes 104 and 106. These meshes 104 and 106 may include thousands or tens of thousands of mesh elements, such as edges, faces, vertices and/or voxels. One or more mesh feature vectors may be computed for one or more mesh elements. The mesh elements and any associated mesh feature vectors may be inputted to the generator. Typically, each of the mesh elements in meshes 104 and 106 can be described by a feature vector having variable size, depending on the features. For instance, when describing a point, the mesh element may be described by a 3-channel vector where the 3 channels describe the X, Y and Z coordinates of a position in three-dimensional space. When describing an edge, the mesh element may be represented by two integers, one for each vertex that defines the edge, where each integer is an index into an array of vertices that makes up the mesh. When describing a face, the mesh element may be represented by three integers, one for each vertex that defines the face. When describing a voxel, the mesh element may be represented by a cubic volume of space. In some implementations, a list of the vertices of a 3D mesh may be supplied to the open source MinkowskiEngine toolkit, which may convert those vertices into voxels for sparce processing. In some implementations, a mesh feature vector may be computed for one ore more mesh elements. A mesh feature is a quantity that describes the attributes (e.g., geometrical and/or structural attributes) of the mesh in the location of the particular mesh elements. In some implementations, only the mesh elements are inputted to the generator. In some implementations, each mesh element is accompanied by an associated mesh element feature vector, such as the feature vectors described in connection with Table 1, above. In addition, the feature vector may contain additional information, such as mesh curvature information (an addition of 3 channels), and edge normal vector information (an additional of a further 3 channels), for a grand total of 9 channels. Still other feature vector compositions are possible, with corresponding channel counts. The 3D meshes discussed so far are only one of several types of 3D representation which may be used to describe the teeth. Other forms of 3D representation include 3D point clouds and voxelized representations.

The U-Net architecture used at step 806 functions by first decreasing the resolution of the input tooth mesh 804, and then by restoring the simplified tooth mesh (i.e., reduced resolution mesh) to the original resolution. This operation enables the information about neighboring teeth (or information about the whole arch) to be captured and integrated into the feature computation. At step 808 of the workflow 800, a feature vector is computed for each element (e.g., each edge) in a high-dimensional space (e.g., 128 channels).

At step 810 of the workflow, for each tooth, the elements with the high-dimensional features are extracted from the output of the encoder-decoder structure. This generates n-tooth edges 812a-812n which are provided to another encoder at step 814 of the workflow 800. The encoder is trained via backpropagation by the high-dimensional features of a given tooth to predict the tooth movement. Backpropagation is a well-established technique for training neural networks, and is known to one who is skilled in the art.

The output of the encoder at step 814 of the workflow 800 is the predicted tooth movements 112 that gets applied to the tooth, to move the tooth into the desired position (for either final setups as described in reference to FIGS. 1-3 or intermediate stages as described in reference to FIGS. 4-6). The encoder at step 814 in workflow 800 is trained via backpropagation to output a transformation for a tooth, regardless of the identity of that tooth. In these implementations, shown in the FIGS., the same encoder is trained to handle each of the teeth present in each of the two arch geometries 106. In other implementations, an encoder may be trained to service a specific tooth or a specific set of teeth. This latter implementation would be reflected in workflow 800 as multiple encoders at step 814 instead of just the one depicted.

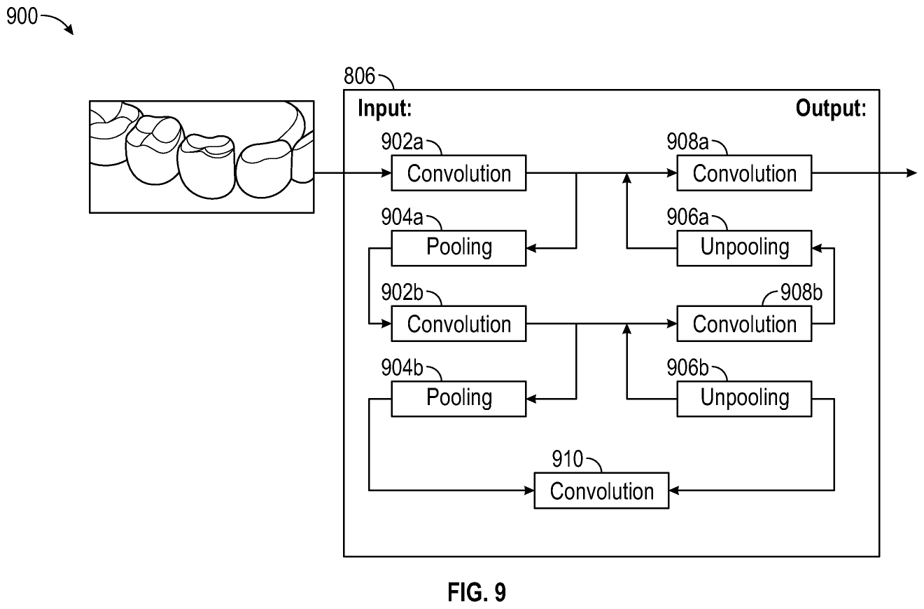

FIG. 9 shows an example U-Net architecture 900 shown in FIG. 8. In general, the U-Net architecture uses a number of pooling layers, such as pooling layers 904a and 904b. The pooling layers in connection with convolution layers, such as convolution layers 902a, 902b, 908a, 908b, and 910, downsample, or shrink, the mesh input. For instance, a downsampling of information in 3D space may take a 3×3×3 set of information and combine it into a single 1×1×1 representation. In the context of 3D mesh information, for example, four neighbor edges of a given edge will be combined into a single edge at the next resolution level. The mesh resolution (mesh surface area) after downsampling will be decreased by a factor of 4×.

According to particular implementations, the convolution layers 902a, 902b, 908b, 908b, and 910 may use edge data to perform mesh convolution. The use of edge information guarantees that the model is not sensitive to different input orders of 3D elements. In addition to or separate from using edge data, convolution layers 902a, 902b, 908b, 908b, and 910 may use vertex data to perform mesh convolution. The use of vertex information is advantageous in that there are typically fewer vertices than edges or faces, so vertex-oriented processing may lead to a lower processing overhead and lower computational cost.

In addition to or separate from using edge data or vertex data, convolution layers 902a, 902b, 908b, 908b, and 910 may use face data to perform mesh convolution. Furthermore, in addition to or separate from using edge data, vertex data, or face data, convolution layers 902a, 902b, 908b, 908b, and 910 may use voxel data to perform mesh convolution. The use of voxel information is advantageous in that, depending on the granularity chosen, there may be significantly fewer voxels to process compared to the vertices, edges or faces in the mesh. Sparse processing (with voxels) may lead to a lower processing overhead and lower computational cost (especially in terms of computer memory or RAM usage).

As described above in reference to FIG. 8, the purpose of the U-Net architecture 900 is to compute a high-dimensional feature vector for the input mesh (which may contain either or both of local and global information about one or more tooth meshes). For instance, according to particular implementations, the U-Net architecture 900 computes a feature vector for each mesh element (e.g., a 128-element feature vector for each edge). This vector exists in a high dimensional space which is capable to represent the local geometry of the edge within the context of the local tooth, and also represent the global geometry of the two arches. The high dimensional features for the elements within each tooth are used by the encoder to predict tooth movement. The accuracy of tooth movement prediction is aided by the combination of this local and global information. The combination of local and global information enables the U-Net architecture 900 to account for geometrical constraints. For example, during the course of the CTA treatment, it is undesirable for teeth to collide in 3D space. The combination of local and global information enables the U-Net architecture 900 to generate transforms which reduce or eliminate the incidence of collisions, and therefore yield greater accuracy relative to prior techniques. Stated another way, one advantage of using mesh element features to train the machine learning model (such as the U-Net architecture 900) over traditional approaches is that the mesh element features provide additional information about at least one of the geometry and the structure of the tooth meshes, which improves the resulting representation(s) generated from the trained U-Net architecture.

The U-Net architecture 900 involves pooling and unpooling operations, which aid the process of extracting mesh element neighbor information. Each successive pooling layer helps the model learn neighbor geometry info by decreasing the resolution, relative to the prior layer. Each successive unpooling layer helps the model expand this summarized neighbor info back to a higher resolution. A sequence of pooling layers following a sequence of unpooling layers will enable the efficient and accurate training of the U-Net and enable the U-Net to output features for each element that contain both local and global geometry info.

While FIG. 9 is depicted with nine total layers, it should be understood that the U-Net architecture 900 can be configured with any number of convolutional layers, any number of pooling layers, and any number of unpooling layers to achieve the desired results.

FIG. 10 shows an example workflow 1000 for the generator 110 shown in either FIG. 1 or FIG. 4. The workflow 1000 is similar to the workflow 800 shown and described in FIG. 8. For instance, both workflows 800 and 1000 produce predicted tooth movements 112. Additionally, and according to particular implementations, the encoder at step 814 of workflow 1000 may also be replaced with multiple encoders, as described above in reference to FIG. 8. For brevity, therefore, each element of workflow 1000 will not be described, and instead only the differences between workflow 800 and workflow 1000 will be mentioned.

Specifically, in step 1002, a pyramid encoder-decoder is used at step 1004 instead of a U-Net architecture that is used at step 806. As should be expected, the pyramid encoder-decoder that is used at step 1004 performs differently than the U-Net architecture that is used at step 806. For instance, the input elements of each tooth mesh (e.g., the edge elements identified in step 804 of the workflow 1000) pass through an encoder structure to generate multiple layers of features in a pyramid. Each successive layer of the encoder has fewer elements, but elements reveal higher dimensional information about the tooth mesh in the feature vector. In other words, each successive layer in the pyramid architecture is configured to reveal higher dimensional information about the tooth mesh. Additionally, an interpolation step is performed at each layer to bring the features from the succession of lower resolutions back into the input resolution of the original tooth mesh. Interpolated features from multiple layers are concatenated and further processed to become high-dimensional features for each mesh element as the output of the pyramid encoder-decoder.

The output of pyramid encoder architecture generated at step 1004 is used by the rest of the workflow 1000 in a similar fashion to how the output of the U-Net architecture generated at step 806 is used by the rest of the workflow 800. Importantly, like workflow 800, the end result of workflow 1000 is the predicted tooth movements 112. This allows for techniques 100 and 400, described above, to be agnostic to the type implementation of generators 110 and 411. This flexibility provides various advantages including, but not limited to, the ability to investigate the accuracy of differently trained U-Net and pyramid architecture-based generators without having to reconfigure the entire system. This may allow, for example, systems implementing techniques 100 and 400 to use one generator 110 or 411 trained on one kind of patient case data received by modules 102 or 402 and another generator 110 or 411 trained on a different kind of patient case data without interruption or degradation of performance of the system. In some implementations, the generator 110 or 411 can is trained to generate tooth movements for all types of teeth (e.g., incisors, cuspids, bicuspids, molars, etc.). In other implementations, one generator 110 or 411 may be trained on only anterior teeth (e.g., incisors and cuspids), and another generator 110 or 411 may be trained on only posterior teeth (e.g., bicuspids and molars). The benefit of this latter approach is improved accuracy, since each of the two generators 110 or 411 is tailored to generate transforms for specific teeth with their own specific geometries.

The U-Net structure in step 806 involves high computer memory usage on account of the fine-grained representation of learned neighbor geometry information for each mesh element. The advantage of the U-Net structure in step 806 is a highly accurate prediction of tooth movements, commensurate with the fine-grained data used for the computation. The Pyramid encoder structure in step 1004 may be used as an alternative in cases where lower memory requirements are present (such as where the computing environment cannot handle the fine-grained data that are involved with the use of the U-Net structure in step 806). A further memory savings can be realized by implementing the alternative structure described above, which replaces the U-Net architecture 900 or pyramid architecture at step 1004 with an encoder.

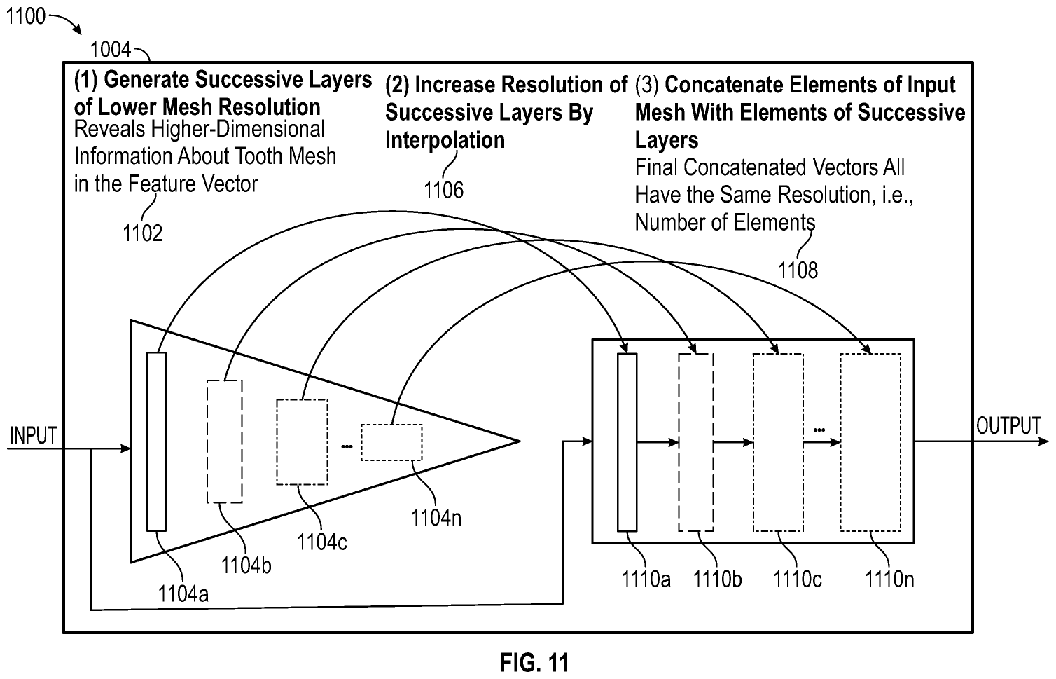

FIG. 11 show an example pyramid encoder-decoder 1100 as shown in FIG. 10. As previously described, the pyramid encoder-decoder has successive layers 1104a-1104n. The manner in which the pyramid architecture 1100 is used during step 1004 of workflow 1000 was also previously described. In particular, in a first step 1102, the pyramid architecture 1100 generates successive layers of lower mesh resolution. This step reveals higher-dimensional information about the tooth mesh and this higher-dimension information is included in a feature vector that the pyramid architecture 1100 produces as output.

Next, is step 1106, the pyramid architecture 1100 increases the resolution of each successive layer of mesh elements (e.g., edges, faces or vertices) using interpolation. For instance, the encoder contained within pyramid architecture 1100 involves successive layers 1110a-1110n, whereby each mesh element is downsampled, to extract information about the mesh at each of the succession of resolutions. Each successive layer attributes additional feature channels to each of the encompassed mesh elements. Each successive layer encompasses a larger portion of the tooth to which the mesh element belongs, and even information about neighboring teeth, information about the whole arch, or even about the two arches as a whole. Interpolation is performed to facilitate the process of concatenating global mesh information from the low-resolution layers with the local mesh information from the high-resolution layers. As is shown, this interpolation results in layers 1110a-1110n. Finally, at step 1108, the pyramid architecture 1100 concatenates elements of the input mesh with elements of successive layers to produce the architecture 1100's output. According to particular implementations, the final concentrated vectors all of the same resolution (i.e., the same number of elements).

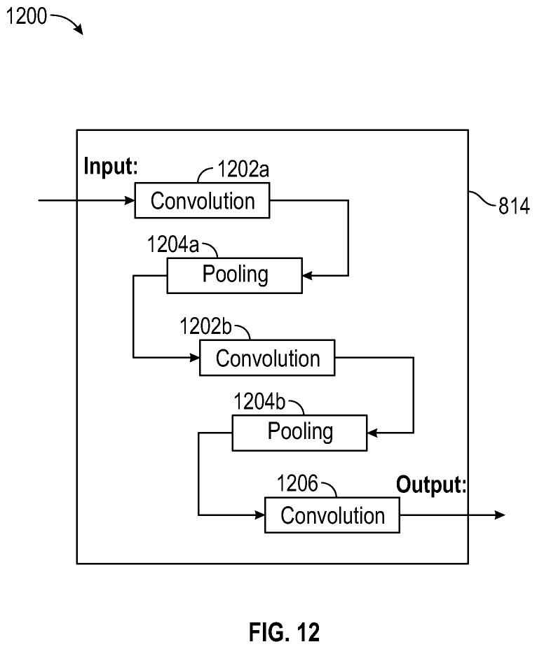

FIG. 12 shows an example encoder 814 shown in FIGS. 8 and 10. As shown, the encoder 814 is a collection of convolution layers 1202a, 1202b, and 1206, and pooling layers 1204a and 1204b. While FIG. 12 is depicted with five total layers, it should be understood that encoder 814 can be configured with any number of layers.

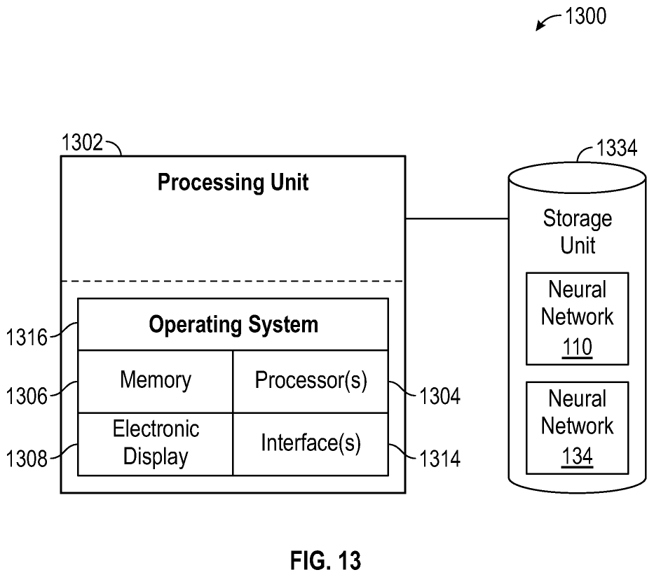

FIG. 13 shows an example processing unit 1302 that operates in accordance with the techniques of the disclosure. The processing unit 1302 provides a hardware environment for the training of one or more of the neural networks described above. For example, the processing unit 1302 may perform techniques 100 and/or 400 to train the neural networks 110 and 134.

In this example, processing unit includes processing circuitry that may include one or more processors 1304 and memory 1306 that, in some examples, provide a computer platform for executing an operating system 1316, which may be a real-time multitasking operating system, for instance, or other type of operating system. In turn, operating system 1316 provides a multitasking operating environment for executing one or more software components such as application 1318. Processors 1304 are coupled to one or more I/O interfaces 1314, which provide I/O interfaces for communicating with devices such as a keyboard, controllers, display devices, image capture devices, other computing systems, and the like. Moreover, the one or more I/O interfaces 1314 may include one or more wired or wireless network interface controllers (NICs) for communicating with a network. Additionally, processors 1304 may be coupled to electronic display 1308.

In this example, processing unit includes processing circuitry that may include one or more processors 1304 and memory 1306 that, in some examples, provide a computer platform for executing an operating system 1316, which may be a real-time multitasking operating system, for instance, or other type of operating system. In turn, operating system 1316 provides a multitasking operating environment for executing one or more software components such as application 1318. Processors 1304 are coupled to one or more I/O interfaces 1314, which provide I/O interfaces for communicating with devices such as a keyboard, controllers, display devices, image capture devices, other computing systems, and the like. Moreover, the one or more I/O interfaces 1314 may include one or more wired or wireless network interface controllers (NICs) for communicating with a network. Additionally, processors 1304 may be coupled to electronic display 1308.

In some examples, processors 1304 and memory 1306 may be separate, discrete components. In other examples, memory 1306 may be on-chip memory collocated with processors 1304 within a single integrated circuit. There may be multiple instances of processing circuitry (e.g., multiple processors 1304 and/or memory 1306) within processing unit 1302 to facilitate executing applications in parallel. The multiple instances may be of the same type, e.g., a multiprocessor system or a multicore processor. The multiple instances may be of different types, e.g., a multicore processor with associated multiple graphics processor units (GPUs). In some examples, processor 1304 may be implemented as one or more microprocessors, digital signal processors (DSPs), application specific integrated circuits (ASICs), field-programmable gate array (FPGAs), or equivalent discrete or integrated logic circuitry, or a combination of any of the foregoing devices or circuitry.

The architecture of processing unit 1302 illustrated in FIG. 13 is shown for example purposes only. Processing unit 1302 should not be limited to the illustrated example architecture. In other examples, processing unit 1302 may be configured in a variety of ways. Processing unit 1302 may be implemented as any suitable computing system, (e.g., at least one server computer, workstation, mainframe, appliance, cloud computing system, and/or other computing system) that may be capable of performing operations and/or functions described in accordance with at least one aspect of the present disclosure. As examples, processing unit 1302 can represent a cloud computing system, server computer, desktop computer, server farm, and/or server cluster (or portion thereof). In other examples, processing unit 1302 may represent or be implemented through at least one virtualized compute instance (e.g., virtual machines or containers) of a data center, cloud computing system, server farm, and/or server cluster. In some examples, processing unit 1302 includes at least one computing device, each computing device having a memory 1306 and at least one processor 1304.

Storage units 1334 may be configured to store information within processing unit 1302 during operation (e.g., geometries 104 and 106, or transformations, 114, or 408). Storage units 1334 may include a computer-readable storage medium or computer-readable storage device. In some examples, storage units 1334 include at least a short-term memory or a long-term memory. Storage units 1334 may include, for example, random access memories (RAM), dynamic random-access memories (DRAM), static random-access memories (SRAM), magnetic discs, optical discs, flash memories, magnetic discs, optical discs, flash memories, or forms of electrically programmable memories (EPROM) or electrically erasable and programmable memories (EEPROM).

In some examples, storage units 1334 are used to store program instructions for execution by processors 1304. Storage units 1334 may be used by software or applications running on processing unit 1302 to store information during program execution and to store results of program execution. For instance, storage units 1334 can store the neural network configurations 110 and 134 as each is being trained using techniques 100 and 400.

While this specification sets forth many specific implementation details, these should not be construed as limitations on the scope of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described components and systems can generally be integrated together in a single system or distributed across multiple systems.

Particular implementations of the of the subject matter have been described. Other implementations are within the scope of the following claims.

1. A computer-implemented method for generating setups for orthodontic alignment treatment, comprising:
receiving, by one or more computer processors, a first digital representation of a patient's teeth, wherein the first digital representation includes a plurality of mesh elements and a respective mesh element feature vector associated with each mesh element in the plurality of mesh elements;
using, by the one or more computer processors and to determine a prediction for one or more tooth movements for a setup, a generator that comprises one or more neural networks and that has been initially trained to predict one or more tooth movements for a setup;
further training, by the one or more computer processors, the generator based on the using, wherein the training of the neural network is modified by performing operations comprising:
predicting, by the generator, one or more tooth movements for a setup based on the first digital representation of the patient's teeth, wherein the one or more tooth movements are described by at least one of a position and an orientation;
quantifying, by the generator, the difference between a representation of the one or more tooth movements predicted by the generator and a representation of one or more reference tooth movements;
generating a loss value based on the quantifying; and
modifying the generator based at least in part on the loss value.

2. The computer-implemented method of claim 1, wherein a mesh element comprises at least one of a vertex, an edge, a face, and a voxel.

3. The computer-implemented method of claim 1, wherein a mesh feature comprises at least one of a spatial feature and a structural feature.

4. The computer-implemented method of claim 1, further comprising producing, by the one or more processors, an output describing one or more transforms to be applied to one or more teeth.

5. The computer-implemented method of claim 4, wherein the setup is an intermediate setup.

6. The computer-implemented method of claim 4, wherein the setup is a final setup.

7. The computer-implemented method of claim 1, wherein modifying the training of the generator comprises adjusting one or more weights of the generator's one or more neural networks.

8. The computer-implemented method of claim 3, wherein the one or more mesh features include vertex XYZ positions, surface normal vectors, and vertex curvatures.

9. The computer-implemented method of claim 1, wherein the generator comprises at least one of a three-dimensional U-Net, a three-dimensional encoder, a three-dimensional decoder, a three-dimensional pyramid encoder/decoder, and a multi-layer perceptron (MLP).

10. The computer-implemented method of claim 1, further comprising generating, by the one or more computer processors, a digital representation of the patient's teeth based on the one or more reference tooth movements.

11. The computer-implemented method of claim 1, wherein the generator is also trained at least in part by a discriminator, which comprises one or more neural networks and has been trained to distinguish between predicted tooth movements and reference tooth movements.

12. A system comprising:
one or more computer processors;
non-transitory computer readable storage having stored thereon a generator that comprises one or more neural networks and that has been initially trained to predict one or more tooth movements for a setup, and instructions that when executed by the one or more processors cause the one or more processors to:
receive a first digital representation of a patient's teeth, wherein the first digital representation includes a plurality of mesh elements and a respective mesh element feature vector associated with each mesh element in the plurality of mesh elements;
use, to determine a prediction for one or more tooth movements for a setup, the generator;
further train the generator based on the using, wherein the training of the neural network is modified by performing operations comprising:
predict, by the generator, one or more tooth movements for a setup based on the first digital representation of the patient's teeth, wherein the one or more tooth movements are described by at least one of a position and an orientation;
quantify, by the generator, the difference between a representation of the one or more tooth movements predicted by the generator and a representation of one or more reference tooth movements;
generate a loss value based on the quantifying; and
modify the generator based at least in part on the loss value.

13. The system of claim 12, wherein a mesh element comprises at least one of a vertex, an edge, a face, and a voxel.

14. The system of claim 12, wherein a mesh feature comprises at least one of a spatial feature and a structural feature.

15. The system of claim 12, where the instructions further cause the one or more processors to produce an output describing one or more transforms to be applied to one or more teeth.

16. The system of claim 15, wherein the setup is an intermediate setup.

17. The system of claim 15, wherein the setup is a final setup.

18. The system of claim 12, wherein modifying the training of the generator comprises adjusting one or more weights of the generator's one or more neural networks.

19. The system of claim 14, wherein the one or more mesh features include vertex XYZ positions, surface normal vectors, and vertex curvatures.

20. The system of claim 12, wherein the generator comprises at least one of a three-dimensional U-Net, a three-dimensional encoder, a three-dimensional decoder, a three-dimensional pyramid encoder/decoder, and a multi-layer perceptron (MLP).
